(19) United States
(12) Patent Application Publication
(10) Pub. No.: US 2026/0128124 A1
(43) Pub. Date: May 7, 2026

(54) METHODS AND SYSTEMS FOR PREDICTION OF NOVEL PATHOGENIC MUTATIONS

(71) Applicant: Foundation Medicine, Inc., Boston, MA (US)

(72) Inventors: Douglas I. LIN, Needham, MA (US); Dean PAVLICK, Cambridge, MA (US); Garrett M. FRAMPTON, Somerville, MA (US); Jonathan Keith KILLIAN, Cambridge, MA (US); James HABERBERGER, Huntsville, NC (US)

(73) Assignee: Foundation Medicine, Inc., Boston, MA (US)

(21) Appl. No.: 19/484,837

(22) PCT Filed: May 14, 2024
(86) PCT No.: PCT/US2024/029298

§ 371 Date: Nov. 14, 2025

Related U.S. Application Data
(60) Provisional application No. 63/466,943, filed on May 16, 2023.

(51) Int. Cl. G16B 20/20; G16B 40/20; G16H 10/60; G16H 15/00; G16H 20/10; G16H 50/70

(52) U.S. Cl. CPC G16B 20/20; G16B 40/20; G16H 10/60; G16H 15/00; G16H 20/10; G16H 50/70

(57) ABSTRACT

Methods and systems for predicting the pathogenicity of variant sequences detected in a sample from a subject are described. The disclosed methods may comprise, for example, receiving sequence read data for a plurality of sequence reads obtained from a sample from a subject; identifying one or more variant sequences based on the sequence read data; providing a variant sequence from the one or more identified variant sequences as input to a trained machine learning model configured to determine a pathogenicity prediction score for the identified variant sequence based on the variant sequence and at least one of additional genomic profiling, demographic pathogenicity prediction score determined for the variant sequence identified in the sample from the subject.

### CROSS-REFERENCE TO RELATED APPLICATION

This application claims the priority benefit of U.S. Provisional Patent Application Ser. No. 63/466,943, filed May 16, 2023, the contents of which are incorporated herein by reference in their entirety.

### FIELD

The present disclosure relates generally to methods and systems for analyzing genomic profiling data, and more specifically to methods and systems for predicting novel pathogenic mutations based on variant sequence data and other genomic or clinical data.

### BACKGROUND

Genomic profiling techniques have enabled research scientists and clinicians to explore and elucidate the landscape of genetic variants that underly a variety of disease states, including a variety of genetic disorders and cancers. Gastrointestinal stromal tumor (GIST), for example, is the most common mesenchymal cancer of the digestive tract. Complete genomic profiling (CGP) and analysis of next generation sequencing (NGS) data using variant calling algorithms has identified several variant forms of the KIT, PDGFRA, NF1, SDHA, and BRAF genes of patients diagnosed with GIST. However, the prevalence of primary driver mutations in these genes varies across samples collected from a large cohort of patients, and furthermore also varies between sample types (e.g., between tissue versus liquid biopsy samples), thus indicating that additional genomic and/or clinical factors also influence the degree to which a mutation in one of these genes is pathogenic. Thus, improved methods for predicting the pathogenicity of genetic mutations based on the detected variant sequences in combination with other genomic and/or clinical data are needed to inform prognosis and treatment selection for patients with genetic disorders and cancers.

### BRIEF SUMMARY OF THE INVENTION

Disclosed herein are methods and systems for predicting the pathogenicity of variant sequences detected in a sample from a subject based on the variant sequence data in combination with other genomic, demographic, and/or clinical data for the subject. The disclosed methods comprise the use of a trained machine learning model that is configured to process input data comprising variant sequence data and at least one of additional genomic profile feature data, demographic feature data, and/or clinical feature data for the sample or subject and output a pathogenicity prediction score for the detected variant sequence. The trained machine learning model can be used to predict novel pathogenic mutations for a given disease, e.g., a given type of cancer. In some embodiments, the trained machine learning model may also be used to predict specific treatment-resistant mutations for the given disease, e.g., a given type of cancer.

In some aspects, disclosed herein are methods for predicting the effects of variant sequences, comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads obtained from the sample from the subject; identifying, using the one or more processors, one or more variant sequences based on the sequence read data; providing, using the one or more processors, a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and outputting, using the one or more processors, the pathogenicity prediction score determined for the variant sequence identified in the sample from the subject.

In some embodiments, the methods disclosed herein can further comprise: comparing, using the one or more processors, the pathogenicity prediction score for the variant sequence identified in the sample from the subject to a predetermined pathogenicity threshold, and based on the comparison: reporting the variant sequence as being pathogenic if its pathogenicity prediction score is greater than or equal to the predetermined pathogenicity threshold; or reporting the variant sequence as being not pathogenic if its pathogenicity prediction score is less than the predetermined pathogenicity threshold. In any of the embodiments herein, the trained machine learning model can be further configured to output a prediction of whether the variant sequence is a drug resistance gene.

In any of the embodiments herein, the subject can be suspected of having or is determined to have cancer. In some embodiments, the cancer can be a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms'tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer can comprise acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In any of the embodiments herein, the disclosed methods can further comprise treating the subject with an anti-cancer therapy. In some embodiments, the anti-cancer therapy can comprise a targeted anti-cancer therapy.

In some embodiments, the targeted anti-cancer therapy can comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In any of the embodiments herein, the disclosed methods can further comprise obtaining the sample from the subject. In any of the embodiments herein, the sample can comprise a tissue biopsy sample, a liquid biopsy sample, or a normal control. In some embodiments, the sample can be a liquid biopsy sample and comprises blood, plasma, cerebrospinal fluid, sputum, stool, urine, or saliva. In some embodiments, the sample can be a liquid biopsy sample and comprises circulating tumor cells (CTCs). In some embodiments, the sample can be a liquid biopsy sample and comprises cell-free DNA (cfDNA), circulating tumor DNA (ctDNA), or any combination thereof.

In any of the embodiments herein, the plurality of nucleic acid molecules can comprise a mixture of tumor nucleic acid molecules and non-tumor nucleic acid molecules. In some embodiments, the tumor nucleic acid molecules can be derived from a tumor portion of a heterogeneous tissue biopsy sample, and the non-tumor nucleic acid molecules can be derived from a normal portion of the heterogeneous tissue biopsy sample. In some embodiments, the sample can comprise a liquid biopsy sample, and the tumor nucleic acid molecules can be derived from a circulating tumor DNA (ctDNA) fraction of the liquid biopsy sample, and the non-tumor nucleic acid molecules can be derived from a non-tumor, cell-free DNA (cfDNA) fraction of the liquid biopsy sample.

In any of the embodiments herein, the one or more adapters can comprise amplification primers, flow cell adaptor sequences, substrate adapter sequences, or sample index sequences. In any of the embodiments herein, the captured nucleic acid molecules can be captured from the amplified nucleic acid molecules by hybridization to one or more bait molecules. In some embodiments, the one or more bait molecules can comprise one or more nucleic acid molecules, each comprising a region that is complementary to a region of a captured nucleic acid molecule. In any of the embodiments herein, the amplifying nucleic acid molecules can comprise performing a polymerase chain reaction (PCR) amplification technique, a non-PCR amplification technique, or an isothermal amplification technique.

In any of the embodiments herein, the sequencing can comprise use of a massively parallel sequencing (MPS) technique, whole genome sequencing (WGS), whole exome sequencing, targeted sequencing, direct sequencing, or Sanger sequencing technique. In some embodiments, the sequencing can comprise massively parallel sequencing, and the massively parallel sequencing technique can comprise next generation sequencing (NGS). In any of the embodiments herein, the sequencer can comprise a next generation sequencer.

In any of the embodiments herein, one or more of the plurality of sequencing reads can overlap one or more gene loci within one or more subgenomic intervals in the sample.

In some embodiments, the one or more gene loci can comprise between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In any of the embodiments herein, the one or more gene loci can comprise ABL1, ACVR1B, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In any of the embodiments herein, the one or more gene loci can comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-16, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In any of the embodiments herein, the disclosed methods can further comprise generating, by the one or more processors, a report indicating the pathogenicity prediction score determined for the variant sequence. In some embodiments, the disclosed methods can further comprise transmitting the report to a healthcare provider. In some embodiments, the report can be transmitted via a computer network or a peer-to-peer connection.

In some aspects, disclosed herein is a method for identifying pathogenic variants comprising: receiving, at one or more processors, sequence read data for a plurality of sequence reads obtained from a sample from a subject; identifying, using the one or more processors, one or more variant sequences based on the sequence read data; providing, using the one or more processors, a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and outputting, using the one or more processors, the pathogenicity prediction score determined for the variant sequence identified in the sample from the subject.

In some embodiments, the disclosed methods can further comprise: comparing, using the one or more processors, the pathogenicity prediction score for the variant sequence identified in the sample from the subject to a predetermined pathogenicity threshold, and based on the comparison: reporting the variant sequence as being pathogenic if its pathogenicity prediction score is greater than or equal to the predetermined pathogenicity threshold; or reporting the variant sequence as being not pathogenic if its pathogenicity prediction score is less than the predetermined pathogenicity threshold. In any of the embodiments herein, the trained machine learning model can be further configured to output a prediction of whether the variant sequence is a drug resistance gene.

In any of the embodiments herein, the disclosed methods can further comprise selecting a treatment for a disease exhibited by the subject based on a pathogenicity prediction score for at least one identified variant sequence that indicates that it is pathogenic. In any of the embodiments herein, the one or more identified variant sequences can comprise one or more single nucleotide substitutions, one or more short insertions, one or more short deletions, or any combination thereof. In any of the embodiments herein, the additional genomic profiling feature data can comprise genomic ancestry, microsatellite instability, tumor mutational burden, a determination of somatic versus germline status for the identified variant sequence, or any combination thereof. In any of the embodiments herein, the additional demographic feature data can comprise the subject's age, sex, race, or any combination thereof. In any of the embodiments herein, the additional clinical feature data can comprise the subject's sample type, disease diagnosis, family history of disease, or any combination thereof.

In any of the embodiments herein, the machine learning model can comprise a supervised machine learning model. In some embodiments, the supervised machine learning model can comprise a random forest model, a gradient boosted decision tree model, an extreme gradient boosted decision tree model, or a support vector machine.

In any of the embodiments herein, the trained machine learning model can be trained using a training dataset that comprises data for variant sequences identified in samples from a cohort of subjects that includes subjects diagnosed with different diseases. In some embodiments, the different diseases can comprise different cancers.

In any of the embodiments herein, the training dataset can further comprise additional genomic profiling feature data for the samples from the cohort of subjects. In some embodiments, the additional genomic profiling feature data can comprise genomic ancestry data, microsatellite instability data, tumor mutational burden data, a determination of somatic versus germline status for the identified variant sequence, or any combination thereof.

In any of the embodiments herein, the training dataset can further comprise additional demographic feature data for the cohort of subjects. In some embodiments, the additional demographic feature data can comprise a subject's age, sex, race, or any combination thereof.

In any of the embodiments herein, the training dataset can further comprise additional clinical feature data for the cohort of subjects. In some embodiments, the additional clinical feature data can comprise a subject's sample type, disease diagnosis, family history of disease, or any combination thereof.

In any of the embodiments herein, the pathogenic prediction score can comprise a real number ranging in value from 0.0 to 1.0. In any of the embodiments herein, the predetermined pathogenicity threshold can have a value ranging from about 0.6 to about 0.9. In any of the embodiments herein, the predetermined pathogenicity threshold can have a value of about 0.75. In any of the embodiments herein, the predetermined pathogenicity threshold can be determined on a per-gene basis.

In any of the embodiments herein, the disease exhibited by the subject can be cancer, and the treatment can be an anti-cancer therapy.

In any of the embodiments herein, the disease exhibited by the subject can be gastrointestinal stromal tumor (GIST), and the treatment can be a tyrosine kinase inhibitor. In some embodiments, the treatment with the tyrosine kinase inhibitor can be recommended if the variant sequence is determined to be pathogenic and is not predicted to be a tyrosine kinase inhibitor resistance gene. In some embodiments, the treatment with the tyrosine kinase inhibitor is not recommended if the variant sequence can be determined to be not pathogenic or can be predicted to be a tyrosine kinase inhibitor resistance gene.

In any of the embodiments herein, the variant sequence can comprise a variant in a BRAF, KIT, NF1, PDGFRA, SDHA, SDHB, SDHC, or SDHD gene. In any of the embodiments herein, the sample can comprise a tissue biopsy sample or a liquid biopsy sample. In some embodiments, the sample can be a liquid biopsy sample and can comprise blood, plasma, cerebrospinal fluid, sputum, stool, urine, or saliva. In some embodiments, the sample can be a liquid biopsy sample and can comprise circulating tumor cells (CTCs).

In some embodiments, the sample can be a liquid biopsy sample and can comprise cell-free DNA (cfDNA), circulating tumor DNA (ctDNA), or any combination thereof.

In some aspects, disclosed herein is a method for selecting a treatment for a subject in need thereof, the method comprising: receiving, at one or more processors, sequence read data for a plurality of sequence reads obtained from a sample from a subject diagnosed with a disease; identifying, using the one or more processors, one or more variant sequences based on the sequence read data; providing, using the one or more processors, a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; comparing, using the one or more processors, the pathogenicity prediction score determined for the variant sequence identified in the sample from the subject to a predetermined pathogenicity threshold to determine if the variant sequence is pathogenic; and selecting a treatment for the disease based on a determination that the variant sequence identified in the sample from the subject is pathogenic.

In some embodiments, the variant sequence can be determined to be pathogenic if its pathogenicity prediction score is greater than or equal to the predetermined pathogenicity threshold. In any of the embodiments herein, the variant sequence can be determined to be not pathogenic if its pathogenicity prediction score is less than the predetermined pathogenicity threshold.

In any of the embodiments herein, the trained machine learning model can be further configured to output a prediction of whether the variant sequence is a drug-resistance gene. In any of the embodiments herein, the additional genomic profiling feature data can comprise genomic ancestry, microsatellite instability, tumor mutational burden, a determination of somatic versus germline status for the identified variant sequence, or any combination thereof.

In any of the embodiments herein, the additional demographic feature data can comprise the subject's age, sex, race, or any combination thereof. In any of the embodiments herein, the additional clinical feature data can comprise the subject's sample type, disease diagnosis, family history of disease, or any combination thereof. In any of the embodiments herein, the disease can be cancer and the treatment can be an anti-cancer therapy.

In any of the embodiments herein, the disease can be gastrointestinal stromal tumor (GIST) and the treatment can be a tyrosine kinase inhibitor. In some embodiments, the treatment with the tyrosine kinase inhibitor can be recommended if the variant sequence is determined to be pathogenic and is not predicted to be a tyrosine kinase inhibitor resistance gene. In some embodiments, the treatment with the tyrosine kinase inhibitor is not recommended if the variant sequence can be determined to be not pathogenic or is predicted to be a tyrosine kinase inhibitor resistance gene. In any of the embodiments herein, the variant sequence can comprise a variant in a BRAF, KIT, NF1, PDGFRA, SDHA, SDHB, SDHC, or SDHD gene.

In any of the embodiments herein, the sample can comprise a tissue biopsy sample or a liquid biopsy sample. In some embodiments, the sample can be a liquid biopsy sample and can comprise blood, plasma, cerebrospinal fluid, sputum, stool, urine, or saliva. In some embodiments, the sample can be a liquid biopsy sample and can comprise circulating tumor cells (CTCs). In some embodiments, the sample can be a liquid biopsy sample and can comprise cell-free DNA (cfDNA), circulating tumor DNA (ctDNA), or any combination thereof.

In some aspects, disclosed herein is a method for classifying variant sequences comprising: receiving, at one or more processors, sequence read data for a plurality of sequence reads obtained from a sample from a subject; identifying, using the one or more processors, at least one variant sequence based on the sequence read data; providing, using the one or more processors, a variant sequence from the one or more identified variant sequences as input to a trained machine learning model configured to determine a cancer type based on the variant sequence and at least one of additional genomic profiling, demographic, or clinical feature data for the sample or subject; and classifying, using the one or more processors, the variant sequence as a driver mutation for the determined cancer type based on the variant sequence and the determined cancer type. In some embodiments, the determined cancer type can be a gastrointestinal stromal tumor (GIST).

In some embodiments, the additional genomic profiling feature data can comprise genomic ancestry, microsatellite instability, tumor mutational burden, a determination of somatic versus germline status for the identified variant sequence, or any combination thereof. In any of the embodiments, the additional demographic feature data can comprise the subject's age, sex, race, or any combination thereof. In any of the embodiments herein, the additional clinical feature data can comprise the subject's sample type, disease diagnosis, family history of disease, or any combination thereof.

In any of the embodiments herein, the one or more identified variant sequences can comprise one or more single nucleotide substitutions, one or more short insertions, one or more short deletions, or any combination thereof. In any of the embodiments herein, the variant sequence can comprise a variant in a BRAF, KIT, NF1, PDGFRA, SDHA, SDHB, SDHC, or SDHD gene.

In any of the embodiments herein, the classification of the variant sequence as a driver mutation for a gastrointestinal stromal tumor (GIST) can be used to diagnose or confirm a diagnosis of GIST in the subject. In any of the embodiments herein, the disclosed methods can further comprise selecting an anti-cancer therapy to administer to the subject based on the classification of the variant sequence as a driver mutation for a gastrointestinal stromal tumor (GIST). In any of the embodiments herein, the disclosed methods can further comprise determining an effective amount of an anti-cancer therapy to administer to the subject based on the classification of the variant sequence as a driver mutation for a gastrointestinal stromal tumor (GIST). In any of the embodiments herein, the disclosed methods can further comprise administering an anti-cancer therapy to the subject based on the classification of the variant sequence as a driver mutation for a gastrointestinal stromal tumor (GIST).

In any of the embodiments herein, the anti-cancer therapy can comprise chemotherapy, radiation therapy, immunotherapy, a targeted therapy, or surgery. In any of the embodiments herein, the anti-cancer therapy can comprise a tyrosine kinase inhibitor.

In some aspects, disclosed herein is a method for diagnosing a disease, the method comprising: diagnosing that a subject has the disease based on a determination of a pathogenicity prediction score for a variant sequence identified in a sample from the subject, wherein the pathogenicity prediction score is determined according to the method of any one of the embodiments herein.

In some aspects, disclosed herein is a method of selecting an anti-cancer therapy, the method comprising: responsive to determining a pathogenicity prediction score for a variant sequence identified in a sample from a subject, selecting an anti-cancer therapy for the subject, wherein the pathogenicity prediction score is determined according to the method of any one of the embodiments herein.

In some aspects, disclosed herein is a method of treating a cancer in a subject, comprising: responsive to determining a pathogenicity score for a variant sequence identified in a sample from the subject, administering an effective amount of an anti-cancer therapy to the subject, wherein the pathogenicity prediction score is determined according to the method of any one of the embodiments disclosed herein.

In any of the embodiments herein, the disclosed methods can further comprise determining, identifying, or applying the value of the pathogenicity prediction score for a variant sequence identified in the sample as a diagnostic value associated with the sample. In any of the embodiments herein, the disclosed methods can further comprise generating a genomic profile for the subject based on the determination of the pathogenicity prediction score for a variant sequence identified in the sample.

In some embodiments, the genomic profile for the subject can further comprise results from a comprehensive genomic profiling (CGP) test, a gene expression profiling test, a cancer hotspot panel test, a DNA methylation test, a DNA fragmentation test, an RNA fragmentation test, or any combination thereof. In any of the embodiments herein, the genomic profile for the subject can further comprise results from a nucleic acid sequencing-based test.

In any of the embodiments herein, the disclosed methods can further comprise selecting an anti-cancer therapy, administering an anti-cancer therapy, or applying an anti-cancer therapy to the subject based on the generated genomic profile. In any of the embodiments herein, the determination of the pathogenicity prediction score for a variant sequence identified in the sample can be used in making suggested treatment decisions for the subject. In any of the embodiments herein, the determination of the pathogenicity prediction score for a variant sequence identified in the sample can be used in applying or administering a treatment to the subject.

In some aspects, disclosed herein is a system comprising: one or more processors; and a memory communicatively coupled to the one or more processors and configured to store instructions that, when executed by the one or more processors, cause the system to: receive sequence read data for a plurality of sequence reads obtained from a sample from a subject; identify one or more variant sequences based on the sequence read data; provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and output the pathogenicity prediction score determined for the variant sequence identified in the sample from the subject.

In some aspects, disclosed herein is a system comprising: one or more processors; and a memory communicatively coupled to the one or more processors and configured to store instructions that, when executed by the one or more processors, cause the system to: receive sequence read data for a plurality of sequence reads obtained from a sample from a subject diagnosed with a disease; identify one or more variant sequences based on the sequence read data; provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; compare the pathogenicity prediction score determined for the variant sequence identified in the sample from the subject to a predetermined pathogenicity threshold to determine if the variant sequence is pathogenic; and select a treatment for the disease based on a determination that the variant sequence identified in the sample from the subject is pathogenic.

In some aspects, disclosed herein is a system comprising: one or more processors; and a memory communicatively coupled to the one or more processors and configured to store instructions that, when executed by the one or more processors, cause the system to: receive sequence read data for a plurality of sequence reads obtained from a sample from a subject; identify at least one variant sequence based on the sequence read data; provide a variant sequence from the one or more identified variant sequences as input to a trained machine learning model configured to determine a cancer type based on the variant sequence and at least one of additional genomic profiling, demographic, or clinical feature data for the sample or subject; and classify the variant sequence as a driver mutation for the determined cancer type based on the variant sequence and determined cancer type. In some embodiments, the determined cancer type can be a gastrointestinal stromal tumor (GIST).

In some aspects, disclosed herein is a non-transitory computer-readable storage medium storing one or more programs, the one or more programs comprising instructions, which when executed by one or more processors of a system, cause the system to: receive sequence read data for a plurality of sequence reads obtained from a sample from a subject; identify one or more variant sequences based on the sequence read data; provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and output the pathogenicity prediction score determined for the variant sequence identified in the sample from the subject.

In some aspects, disclosed herein is a non-transitory computer-readable storage medium storing one or more programs, the one or more programs comprising instructions, which when executed by one or more processors of a system, cause the system to: receive sequence read data for a plurality of sequence reads obtained from a sample from a subject diagnosed with a disease; identify one or more variant sequences based on the sequence read data; provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; compare the pathogenicity prediction score determined for the variant sequence identified in the sample from the subject to a predetermined pathogenicity threshold to determine if the variant sequence is pathogenic; and select a treatment for the disease based on a determination that the variant sequence identified in the sample from the subject is pathogenic.

In some aspects, disclosed herein is a non-transitory computer-readable storage medium storing one or more programs, the one or more programs comprising instructions, which when executed by one or more processors of a system, cause the system to: receive sequence read data for a plurality of sequence reads obtained from a sample from a subject; identify at least one variant sequence based on the sequence read data; provide a variant sequence from the one or more identified variant sequences as input to a trained machine learning model configured to determine a cancer type based on the variant sequence and at least one of additional genomic profiling, demographic, or clinical feature data for the sample or subject; and classify the variant sequence as a driver mutation for a gastrointestinal stromal tumor (GIST) based on the variant sequence and determined cancer type.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for predicting a pathogenicity score for a variant sequence detected in a sample from a subject based on the variant sequence in combination with at least one of additional genomic profiling, demographic, or clinical feature data for the sample or subject, according to one implementation of the methods described herein.

FIG. 2 provides a non-limiting example of a process flowchart for selecting a treatment for disease, according to one implementation of the methods described herein.

FIG. 3 provides a non-limiting example of a process flowchart for classifying a variant sequence as a driver mutation for a gastrointestinal stromal tumor (GIST), according to one implementation of the methods described herein.

FIG. 4 depicts an exemplary computing device or system in accordance with one embodiment of the present disclosure.

FIG. 5 depicts an exemplary computer system or computer network, in accordance with some instances of the systems described herein.

FIG. 6 provides a non-limiting schematic comparison between: (i) a conventional process for assigning functional status and reporting a variant sequence as being pathogenic, and (ii) a novel process for assigning functional status based on the use of a trained machine learning classifier and reporting the variant sequence as being pathogenic.

### DETAILED DESCRIPTION

Disclosed herein are methods and systems for predicting the pathogenicity of variant sequences detected in a sample from a subject based on the variant sequence data in combination with other genomic, demographic, and/or clinical data for the subject. The disclosed methods comprise the use of a trained machine learning model that is configured to process input data comprising variant sequence data and at least one of additional genomic profile feature data, demographic feature data, and/or clinical feature data for the sample or subject and output a pathogenicity prediction score for the detected variant sequence. The trained machine learning model can be used to predict novel pathogenic mutations for a given disease, e.g., a given type of cancer. In some embodiments, the trained machine learning model may also be used to predict specific treatment-resistant mutations for the given disease, e.g., a given type of cancer.

In some instances, for example, methods for identifying pathogenic variants are described that comprise: receiving sequence read data for a plurality of sequence reads obtained from a sample from a subject; identifying one or more variant sequences based on the sequence read data; providing a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and outputting the pathogenicity prediction score determined for the variant sequence identified in the sample from the subject.

In some instances, methods for selecting a treatment for a subject in need thereof are described that comprise: receiving sequence read data for a plurality of sequence reads obtained from a sample from a subject diagnosed with a disease; identifying one or more variant sequences based on the sequence read data; providing a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; comparing the pathogenicity prediction score determined for the variant sequence identified in the sample from the subject to a predetermined pathogenicity threshold to determine if the variant sequence is pathogenic; and selecting a treatment for the disease based on a determination that the variant sequence identified in the sample from the subject is pathogenic.

In some instances, methods for classifying variant sequences are described that comprise: receiving sequence read data for a plurality of sequence reads obtained from a sample from a subject; identifying at least one variant sequence based on the sequence read data; providing a variant sequence from the one or more identified variant sequences as input to a trained machine learning model configured to determine a cancer type based on the variant sequence and at least one of additional genomic profiling, demographic, or clinical feature data for the sample or subject; and classifying the variant sequence as a driver mutation for a gastrointestinal stromal tumor (GIST) based on the variant sequence and determined cancer type.

In some instances, the machine learning model may comprise a supervised machine learning model. In some instances, the supervised machine learning model may comprise a random forest model, a gradient boosted decision tree model, an extreme gradient boosted decision tree model, or a support vector machine.

In some instances, the trained machine learning model is trained using a training dataset that comprises data for variant sequences identified in samples from a cohort of subjects that includes subjects diagnosed with different diseases. In some instances, the different diseases comprise different cancers.

In some instances, the training dataset used to train the machine learning model may further comprise additional genomic profiling feature data, demographic feature data, and/or clinical feature data for the samples from or subjects in the cohort of subjects.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Prediction of Novel Pathogenic Mutations

As noted above, genomic profiling techniques have enabled research scientists and clinicians to explore and elucidate the landscape of genetic variants that underly a variety of disease states, including a variety of genetic disorders and cancers. Gastrointestinal stromal tumor (GIST), for example, is the most common mesenchymal cancer of the digestive tract. Beyond surgery, treatment for GIST focuses largely on tyrosine kinase inhibitors (TKI), the selection of which (and potential resistance to) depends on the presence of select mutations.

Complete genomic profiling (CGP) and analysis of next generation sequencing (NGS) data using variant calling algorithms has identified several variant forms of the KIT, PDGFRA, NF1, SDHA, and BRAF genes of patients diagnosed with GIST. However, the prevalence of primary driver mutations in these genes varies across samples collected from a large cohort of patients, and furthermore also varies between sample types (e.g., between tissue versus liquid biopsy samples), thus indicating that additional genomic and/or clinical factors also influence the degree to which a mutation in one of these genes is pathogenic.

For example, variant identification and evaluation of other genomic profiling metrics (e.g., microsatellite instability (MSI) and tumor mutational burden) in a recent study of tissue samples from GIST patients (2,198 samples in total) identified the following prevalence of primary driver mutations: KIT (77%), PDGFRA (8%), NF1 (6%), SDHA (2%) and BRAF (1%). Rates of molecular markers previously associated with worse prognosis included: CDKN2A/2B (29%), RB1 (9%), MTAP (7%), and TP53 (6%). Tumors were microsatellite stable (98%) and exhibited low TMB (99.5%).

Variant identification and evaluation of other genomic profiling metrics for liquid biopsy samples from GIST patients (150 samples in total) indicated that KIT and PGFRA alterations were present overall in 45% and 2% of cases, respectively. By stratifying the cohort based on tumor fraction (TF), KIT and PDGFRA mutations were present in 77% and 8% of cases, respectively, when tumor fraction (TF) was >10%. In the liquid biopsy cohort, 58% (39 out of 67) of KIT-mutant samples had a co-occurring imatinib-resistant KIT alteration. In addition, 4 of 150 patients (3%) were predicted to harbor a germline KIT mutation, including one patient (0.6%) with a potential imatinib-resistant KIT D820G germline mutation, and another patient with clinical suspicion of germline KIT L576P mutation due to the presence of multiple primary GISTs, hyperplasia of myenteric plexus, and dysplastic skin nevi.

Sequence data for a cohort of 27 paired tissue and liquid biopsy samples from the same GIST patients was also analyzed, and demonstrated concordance of the identified driver mutations in samples from 12 of 27 patients. There was no detectable circulating tumor DNA (ctDNA) in the liquid biopsy samples for which a driver alteration was not detected (TF<1%).

Collectively, these data confirm that additional genomic profile features (e.g., MSI, TMB, TF, etc.), and/or other demographic or clinical factors may influence the degree to which a mutation in one of the KIT, PDGFRA, NF1, SDHA, or BRAF genes is pathogenic.

Applying a pan-cancer computational algorithm, as described below, to the analysis of the CGP/sequencing data for GIST patient samples predicted several novel KIT, PDGFRA and SDHB pathogenic mutations which have not been previously reported in the literature. The computational algorithm may be used to predict pathogenic mutations, e.g., pathogenic driver mutations, for GIST in the BRAF, KIT, NF1, PDGFRA, and SDHA/B/C/D genes (or to predict pathogenic mutations in other driver genes for other cancers), and to predict gene alterations that may confer tyrosine kinase resistance in GIST (or gene alterations that may confer drug resistance for other cancer treatments for other tumor types). In addition, a subset of SDHA/B/C/D or NF1 mutations may be both pathogenic and of germline origin, which may predispose patients to the development of GIST and/or other tumor types.

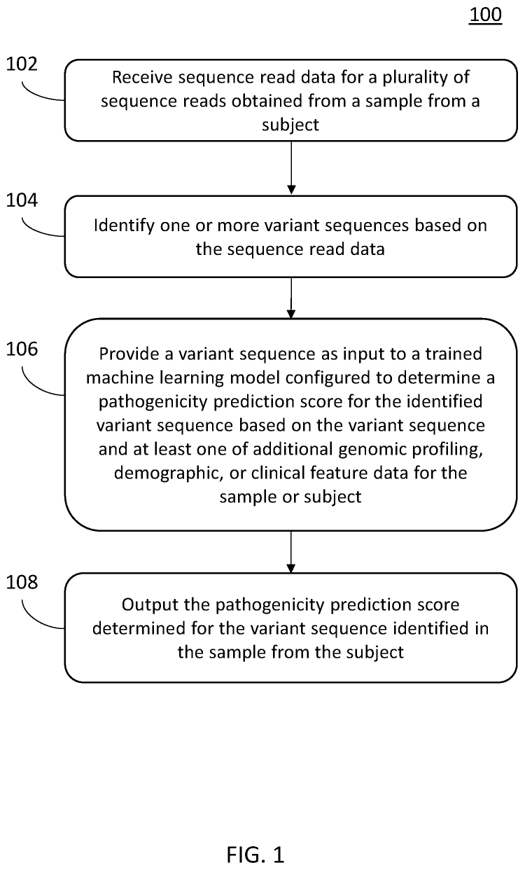

FIG. 1 provides a non-limiting example of a flowchart for a process 100 for predicting a pathogenicity score for a variant sequence detected in a sample from a subject based on the variant sequence in combination with at least one of additional genomic profiling, demographic, and/or clinical feature data for the subject. Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, sequence read data for a plurality of sequence reads obtained from a sample from a subject (e.g., a patient) is received. In some instances, the sample may comprise, e.g., a tissue biopsy sample, a liquid biopsy sample, and/or a normal (healthy) control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the plurality of sequence reads may be derived from a targeted sequencing technique, e.g., a targeted exome sequencing technique. In some instances, the sequence read data may be derived from, e.g., a whole genome or whole exome sequencing technique, as opposed to a targeted exome sequencing technique, to increase the number of genomic features (e.g., the number of short variants) detected.

In some instances, the sequence read data for the plurality of sequence reads may comprise data for aligned sequence reads (e.g., sequence reads that have been aligned to a reference genome such as the human reference genome HG38) and may be received as a BAM file by a system configured to perform the methods described herein.

At step 104 in FIG. 1, one or more variant sequences may be identified based on the sequence read data. In some instances, for example, variant sequences may be identified by identifying genomic positions where the nucleotides in the aligned sequence reads differ from those in the reference genome. In some instances, variant sequences may be identified by aligning the plurality of sequence reads to a reference genome, if not already aligned, and identifying genomic positions where the nucleotides in the aligned sequence reads differ from those in the reference genome.

In some instances, the one or more identified variant sequences may comprise one or more single nucleotide substitutions, one or more short indels (e.g., one or more short insertions, one or more short deletions), or any combination thereof. In some instances, the one or more identified variant sequences may comprise substitutions, insertions, or deletions ranging in length from 1 to about 50 base pairs (bp). In some instances, the one or more identified variant sequences may comprise a variant sequence of 1, 2, 3, 4, 5, 6, 7, 8, 9, 10, 15, 20, 25, 30, 35, 40, 45, 50, or more than 50 bp in length, or of any value within this range.

At step 106 in FIG. 1, a variant sequence (e.g., one or more of the variant sequences identified based on the sequence read data) is provided as input to a trained machine learning model configured to determine a pathogenicity prediction score for the identified variant sequence based on the variant sequence and at least one of additional genomic profiling, demographic, or clinical feature data for the sample or subject.

In some instances, the additional genomic profiling feature data may comprise, for example, genomic ancestry, microsatellite instability, tumor mutational burden, a determination of somatic versus germline status for the identified variant sequence, or any combination thereof. In some instances, the additional genomic profiling feature may also be determined based on the sequence read data received for the sample.

In some instances, the additional demographic feature data may comprise, for example, the subject's age, sex, race, disease diagnosis, family history of disease, or any combination thereof.

In some instances, the additional clinical feature data may comprise, for example, the subject's sample type, disease diagnosis, family history of disease, or any combination thereof.

In some instances, the machine learning model may comprise, for example, a supervised, semi-supervised, or unsupervised machine learning model. In some instances, the model may comprise a supervised machine learning model. In some instances, the supervised machine learning model may comprise a random forest model, a gradient boosted decision tree model, an extreme gradient boosted decision tree model, or a support vector machine.

In some instances, the trained machine learning model may be trained using a training dataset that comprises data for variant sequences identified in samples from a cohort of subjects that have been diagnosed with a specific disease, for example, a specific cancer. In some instances, the trained machine learning model may be trained using a training dataset that comprises data for variant sequences identified in samples from a cohort of subjects that includes subjects diagnosed with different diseases, for example, different cancers.

In some instances, the training dataset may further comprise additional genomic profiling feature data for the samples from the cohort of subjects. For example, the additional genomic profiling feature data may comprise genomic ancestry data, microsatellite instability data, tumor mutational burden data, a determination of somatic versus germline status for the identified variant sequence, or any combination thereof.

In some instances, the training dataset may further comprise additional demographic feature data for the cohort of subjects from which the samples were collected. For example, the additional demographic feature data may comprise a subject's age, sex, race, or any combination thereof.

In some instances, the training dataset may further comprise additional clinical feature data for the cohort of subjects. For example, the additional clinical feature data may comprise a subject's sample type, disease diagnosis, family history of disease, or any combination thereof.

In some instances, the training dataset may comprise data from a pan-cancer analysis. For example, a training dataset for training a machine learning model to predict pathogenic driver mutations for gastrointestinal stromal tumors (GIST) has been developed based on data for over 500,000 tumor samples of genes that may be enriched in advanced GIST. The trained model provides a pathogenicity prediction score based on many genomic profiling features and/or demographic characteristics, including tumor type bias.

In some instances, the pathogenic prediction score may comprise a real number ranging in value from 0.0 to 1.0. In some instances, for example, the pathogenicity score may have a value of 0.0, 0.1, 0.2, 0.3, 0.4, 0.5, 0.6, 0.7, 0.8, 0.9, or 1.0. In some instances, the pathogenicity score may have any value within this range.

At step 108 in FIG. 1, the pathogenicity prediction score determined for the variant sequence identified in the sample from the subject may be output.

In some instances, the process depicted in FIG. 1 may further comprise comparing the pathogenicity prediction score for the variant sequence identified in the sample from the subject to a predetermined pathogenicity threshold and, based on the comparison, reporting the variant sequence as being pathogenic if its pathogenicity prediction score is greater than or equal to the predetermined pathogenicity threshold; or reporting the variant sequence as being not pathogenic if its pathogenicity prediction score is less than the predetermined pathogenicity threshold.

The pathogenicity threshold may be determined by any of a variety of methods known to those of skill in the art. For example, in some instances the pathogenicity threshold may be determined based on an analysis of the genomic profiling features, demographic characteristics, and/or clinical data for the cohort of subjects for which the training data used to train the machine learning model was derived. In some instances, the pathogenicity threshold may be based on the distribution of pathogenicity scores. For example, if the distribution of pathogenicity scores is bimodal, a threshold may be determined that distinguishes between the pathogenicity score data in the two modes. In some instances, the pathogenicity threshold may be determined based on expert adjudication and domain knowledge. In some instances, the predetermined pathogenicity threshold may have a value ranging from about 0.6 to about 0.9. In some instances, the predetermined pathogenicity threshold may have a value of about 0.75. In some instances, the predetermined pathogenicity threshold may have a value of about 0.50, 0.55, 0.60, 0.65, 0.70, 0.75, 0.80, 0.85, 0.90, or any value within this range of values. In some instances, the predetermined pathogenicity threshold may be determined on a per-gene basis, i.e., the pathogenicity threshold may be difference for different genes within which the identified variant sequence occurs.

In some instances, the trained machine learning model may be further configured to output a prediction of whether the variant sequence is a drug resistance gene. For example, in some instances, the trained machine learning model may be configured to output a prediction of whether a variant sequence reported to be pathogenic is also a drug resistance gene.

In some instances, process 100 may further comprise selecting a treatment for a disease (e.g., a cancer) exhibited by the subject based on a pathogenicity prediction score for at least one identified variant sequence that indicates that it is pathogenic. For example, in some instances the disease exhibited by the subject may be cancer, and the treatment may be an anti-cancer therapy.

In some instances, the disease exhibited by the subject may be gastrointestinal stromal tumor (GIST), and the treatment may be a tyrosine kinase inhibitor. In some instances, treatment with the tyrosine kinase inhibitor may be recommended if the variant sequence is determined to be pathogenic and is not predicted to be a tyrosine kinase inhibitor resistance gene. In some instances, treatment with the tyrosine kinase inhibitor may not be recommended if the variant sequence is determined to be not pathogenic or is predicted to be a tyrosine kinase inhibitor resistance gene.

In some instances, the disease exhibited by the subject may be gastrointestinal stromal tumor (GIST), and the variant sequence (e.g., pathogenic variant sequence) may comprise a variant (e.g., a driver mutation) in the BRAF, KIT, NF1, PDGFRA, SDHA, SDHB, SDHC, or SDHD gene.

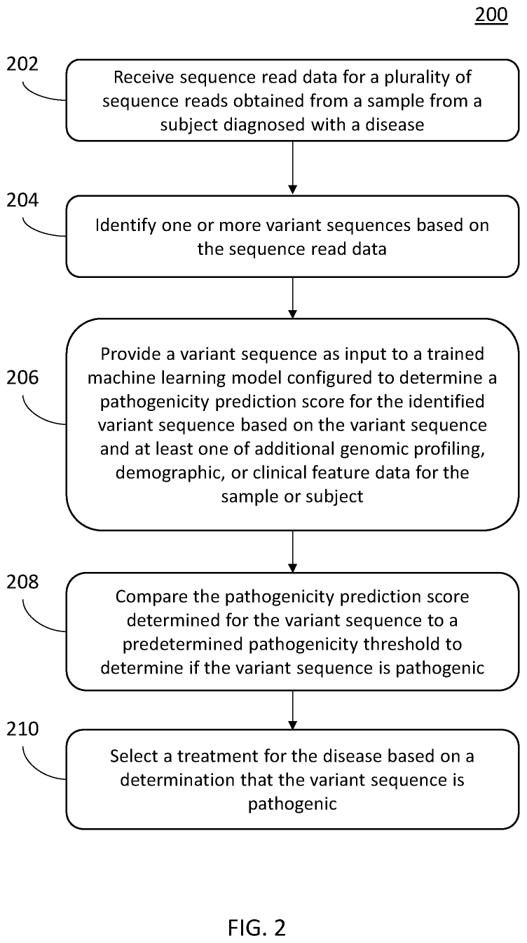

FIG. 2 provides a non-limiting example of a flowchart for a process 200 for selecting a treatment for disease based on a pathogenic prediction score determined for a variant sequence detected in a sample from a subject. Process 200 can be performed, for example, using one or more electronic devices implementing a software platform. In some examples, process 200 is performed using a client-server system, and the blocks of process 200 are divided up in any manner between the server and a client device. In other examples, the blocks of process 200 are divided up between the server and multiple client devices. Thus, while portions of process 200 are described herein as being performed by particular devices of a client-server system, it will be appreciated that process 200 is not so limited. In other examples, process 200 is performed using only a client device or only multiple client devices. In process 200, some blocks are, optionally, combined, the order of some blocks is, optionally, changed, and some blocks are, optionally, omitted. In some examples, additional steps may be performed in combination with the process 200. Accordingly, the operations as illustrated (and described in greater detail below) are exemplary by nature and, as such, should not be viewed as limiting.

At step 202 in FIG. 2, sequence read data for a plurality of sequence reads obtained from a sample from a subject (e.g., a patient) diagnosed with a disease is received. In some instances, the sample may comprise, e.g., a tissue biopsy sample, a liquid biopsy sample, and/or a normal (healthy) control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the plurality of sequence reads may be derived from a targeted sequencing technique, e.g., a targeted exome sequencing technique. In some instances, the sequence read data may be derived from, e.g., a whole genome or whole exome sequencing technique, as opposed to a targeted exome sequencing technique, to increase the number of genomic features (e.g., the number of short variants) detected.

In some instances, the sequence read data for the plurality of sequence reads may comprise data for aligned sequence reads (e.g., sequence reads that have been aligned to a reference genome such as the human reference genome HG38) and may be received as a BAM file by a system configured to perform the methods described herein.

At step 204 in FIG. 2, one or more variant sequences may be identified based on the sequence read data. In some instances, for example, variant sequences may be identified by identifying genomic positions where the nucleotides in the aligned sequence reads differ from those in the reference genome. In some instances, variant sequences may be identified by aligning the plurality of sequence reads to a reference genome, if not already aligned, and identifying genomic positions where the nucleotides in the aligned sequence reads differ from those in the reference genome.

In some instances, the one or more identified variant sequences may comprise one or more single nucleotide substitutions, one or more short indels (e.g., one or more short insertions, one or more short deletions), or any combination thereof. In some instances, the one or more identified variant sequences may comprise substitutions, insertions, or deletions ranging in length from 1 to about 50 base pairs (bp). In some instances, the one or more identified variant sequences may comprise a variant sequence of 1, 2, 3, 4, 5, 6, 7, 8, 9, 10, 15, 20, 25, 30, 35, 40, 45, 50, or more than 50 bp in length, or of any value within this range.

At step 206 in FIG. 2, a variant sequence (e.g., one or more of the variant sequences identified based on the sequence read data) is provided as input to a trained machine learning model configured to determine a pathogenicity prediction score for the identified variant sequence based on the variant sequence and at least one of additional genomic profiling, demographic, or clinical feature data for the sample or subject.

In some instances, the additional genomic profiling feature data may comprise, for example, genomic ancestry, microsatellite instability, tumor mutational burden, a determination of somatic versus germline status for the identified variant sequence, or any combination thereof. In some instances, the additional genomic profiling feature data may also be determined based on the sequence read data received for the sample.

In some instances, the additional demographic feature data may comprise, for example, the subject's age, sex, race, or any combination thereof.

In some instances, the additional clinical feature data may comprise, for example, the subject's sample type, disease diagnosis, family history of disease, or any combination thereof.

As described above with respect to step 106 in FIG. 1, the machine learning model may comprise, for example, a supervised, semi-supervised, or unsupervised machine learning model. In some instances, the model may be a supervised machine learning model (e.g., a random forest model, a gradient boosted decision tree model, an extreme gradient boosted decision tree model, or a support vector machine), and may be trained as described above with respect to step 106 in FIG. 1.

In some instances, the trained machine learning model may be further configured to output a prediction of whether the identified variant sequence is a drug-resistance gene.

Again, the pathogenic prediction score may comprise, for example, a real number ranging in value from 0.0 to 1.0. In some instances, for example, the pathogenicity score may have a value of 0.0, 0.1, 0.2, 0.3, 0.4, 0.5, 0.6, 0.7, 0.8, 0.9, or 1.0. In some instances, the pathogenicity score may have any value within this range.

At step 208 in FIG. 2, the pathogenicity prediction score determined for the variant sequence may be compared to a predetermined pathogenicity threshold to determine if the variant sequence is pathogenic. In some instances, the variant sequence may be determined to be pathogenic if its pathogenicity prediction score is greater than or equal to the predetermined pathogenicity threshold. In some instances, the variant sequence may be determined to be not pathogenic if its pathogenicity prediction score is less than the predetermined pathogenicity threshold.

At step 210 in FIG. 2, a treatment for the disease may be recommended and/or selected based on a determination that the variant sequence is pathogenic. For example, based on a determination that the variant sequence is pathogenic, a treatment for the disease may be recommended by the system. Alternatively, based on a determination that the variant sequence is pathogenic, a treatment for the disease may be selected, e.g., by a physician or healthcare provider. In some instances, the disease may be cancer, and the treatment may be an anti-cancer therapy.

In some instances, the disease may be gastrointestinal stromal tumor (GIST), and the treatment may be, e.g., a tyrosine kinase inhibitor. Treatment with a tyrosine kinase inhibitor may be recommended if the variant sequence is determined to be pathogenic and is not predicted to be a tyrosine kinase inhibitor resistance gene. In some instances, treatment with a tyrosine kinase inhibitor may not be recommended, for example, if the variant sequence is determined to be not pathogenic or is predicted to be a tyrosine kinase inhibitor resistance gene.

In some instances of gastrointestinal stromal tumor (GIST), the variant sequence (e.g., pathogenic variant sequence) may comprise a variant (e.g., a driver mutation) in a BRAF, KIT, NF1, PDGFRA, SDHA, SDHB, SDHC, or SDHD gene.

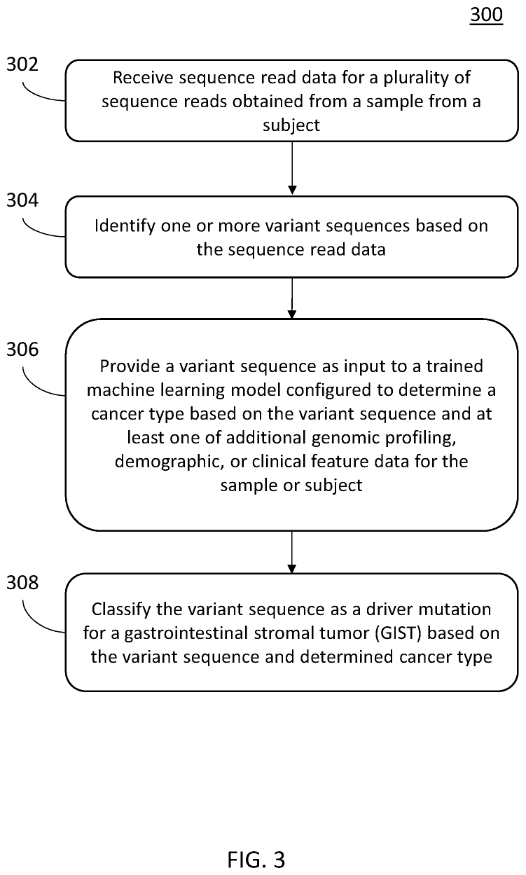

FIG. 3 provides a non-limiting example of a flowchart for a process 300 for classifying a variant sequence as a driver mutation for a gastrointestinal stromal tumor (GIST). Process 300 can be performed, for example, using one or more electronic devices implementing a software platform. In some examples, process 300 is performed using a client-server system, and the blocks of process 300 are divided up in any manner between the server and a client device. In other examples, the blocks of process 300 are divided up between the server and multiple client devices. Thus, while portions of process 300 are described herein as being performed by particular devices of a client-server system, it will be appreciated that process 300 is not so limited. In other examples, process 300 is performed using only a client device or only multiple client devices. In process 300, some blocks are, optionally, combined, the order of some blocks is, optionally, changed, and some blocks are, optionally, omitted. In some examples, additional steps may be performed in combination with the process 300. Accordingly, the operations as illustrated (and described in greater detail below) are exemplary by nature and, as such, should not be viewed as limiting.

At step 302 in FIG. 3, sequence read data for a plurality of sequence reads obtained from a sample from a subject (e.g., a patient) diagnosed with a disease is received. In some instances, the sample may comprise, e.g., a tissue biopsy sample, a liquid biopsy sample, and/or a normal (healthy) control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

As indicated above with regard to FIG. 1 and FIG. 2, in some instances the plurality of sequence reads may be derived from a targeted sequencing technique, e.g., a targeted exome sequencing technique. In some instances, the sequence read data may be derived from, e.g., a whole genome or whole exome sequencing technique, as opposed to a targeted exome sequencing technique, to increase the number of genomic features (e.g., the number of short variants) detected.

In some instances, the sequence read data for the plurality of sequence reads may comprise data for aligned sequence reads (e.g., sequence reads that have been aligned to a reference genome such as the human reference genome HG38) and may be received as a BAM file by a system configured to perform the methods described herein.

At step 304 in FIG. 3, one or more variant sequences may be identified based on the sequence read data. In some instances, for example, variant sequences may be identified by identifying genomic positions where the nucleotides in the aligned sequence reads differ from those in the reference genome. In some instances, variant sequences may be identified by aligning the plurality of sequence reads to a reference genome, if not already aligned, and identifying genomic positions where the nucleotides in the aligned sequence reads differ from those in the reference genome.

In some instances, the one or more identified variant sequences may comprise one or more single nucleotide substitutions, one or more short indels (e.g., one or more short insertions, one or more short deletions), or any combination thereof. In some instances, the one or more identified variant sequences may comprise substitutions, insertions, or deletions ranging in length from 1 to about 50 base pairs (bp). In some instances, the one or more identified variant sequences may comprise a variant sequence of 1, 2, 3, 4, 5, 6, 7, 8, 9, 10, 15, 20, 25, 30, 35, 40, 45, 50, or more than 50 bp in length, or of any value within this range.

At step 306 in FIG. 3, a variant sequence (e.g., one or more of the variant sequences identified based on the sequence read data) is provided as input to a trained machine learning model configured to determine a cancer type based on the identified variant sequence and at least one of additional genomic profiling, demographic, or clinical feature data for the sample or subject.

In some instances, the additional genomic profiling feature data may comprise, for example, genomic ancestry, microsatellite instability, tumor mutational burden, a determination of somatic versus germline status for the identified variant sequence, or any combination thereof. In some instances, the additional genomic profiling feature data may also be determined based on the sequence read data received for the sample.

In some instances, the additional demographic feature data may comprise, for example, the subject's age, sex, race, or any combination thereof.

In some instances, the additional clinical feature data comprises the subject's sample type, disease diagnosis, family history of disease, or any combination thereof.

The machine learning model may comprise, for example, a supervised, semi-supervised, or unsupervised machine learning model. Examples of suitable supervised machine learning models include, but are not limited to, random forest models, gradient boosted decision tree models, extreme gradient boosted decision tree models, or support vector machines.

In some instances, the trained machine learning model may be trained using a training dataset that comprises data for variant sequences identified in samples from a cohort of subjects that have been diagnosed with a specific disease, for example, a specific cancer. In some instances, the trained machine learning model may be trained using a training dataset that comprises data for variant sequences identified in samples from a cohort of subjects that includes subjects diagnosed with different diseases, for example, different cancers.

In some instances, the training dataset may further comprise additional genomic profiling feature data for the samples from the cohort of subjects. For example, the additional genomic profiling feature data may comprise genomic ancestry data, microsatellite instability data, tumor mutational burden data, a determination of somatic versus germline status for the identified variant sequence, or any combination thereof.

In some instances, the training dataset may further comprise additional demographic feature data for the cohort of subjects from which the samples were collected. For example, the additional demographic feature data may comprise a subject's age, sex, race, or any combination thereof.

In some instances, the training dataset may further comprise additional clinical feature data for the cohort of subjects. For example, the additional clinical feature data may comprise a subject's sample type, disease diagnosis, family history of disease, or any combination thereof.

In some instances, the training dataset may comprise data from a pan-cancer analysis. For example, a training dataset for training a machine learning model to predict pathogenic driver mutations for gastrointestinal stromal tumors (GIST) has been developed based on data for over 500,000 tumor samples of genes that may be enriched in advanced GIST. The trained model provides a pathogenicity prediction score based on many genomic profiling features and/or demographic characteristics, including tumor type bias.

At step 308 in FIG. 3, the variant sequence is classified as a driver mutation for a gastrointestinal stromal tumor (GIST) based on the variant sequence and the determined cancer type. In some instances, the variant sequence (e.g., pathogenic variant) may comprises a variant (e.g., a driver mutation) in a BRAF, KIT, NF1, PDGFRA, SDHA, SDHB, SDHC, or SDHD gene.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for predicting the pathogenicity of a variant sequence identified in a sample from a subject may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for predicting the pathogenicity of a variant sequence identified in a sample from a subject may be used to select a subject (e.g., a patient) for a clinical trial based on the predicted pathogenicity score determined for mutations detected at one or more gene loci. In some instances, patient selection for clinical trials based on, e.g., prediction of pathogenic mutations identified at one or more gene loci, may accelerate the development of targeted therapies and improve the healthcare outcomes for treatment decisions.

In some instances, the disclosed methods for predicting the pathogenicity of a variant sequence identified in a sample from a subject may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for predicting the pathogenicity of a variant sequence identified in a sample from a subject may be used in treating a disease (e.g., a cancer) in a subject. For example, in response to predicting that a mutation identified in a sample from the subject is pathogenic using any of the methods disclosed herein, an effective amount of an anti-cancer therapy or anti-cancer treatment may be administered to the subject.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a prediction of pathogenicity or response to a change in the prediction of pathogenicity for a variant sequencing identified in the subject.

In some instances, the pathogenicity score for one or more variant sequences identified in a sample from a subject may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for predicting the pathogenicity of a variant sequence identified in a sample from a subject may be implemented as part of a genomic profiling process that comprises identification of the presence of variant sequences at one or more gene loci in a sample derived from the subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for predicting the pathogenicity of a variant sequence identified in a sample from a subject as part of a genomic profiling process (or inclusion of the output from the disclosed methods for predicting the pathogenicity of a variant sequence identified in a sample from a subject as part of the genomic profile of the subject) can improve the validity of, e.g., disease detection calls and treatment decisions, made on the basis of the genomic profile by, for example, independently confirming the presence of a pathogenic mutation in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms'tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature # TM 333, Jan. 1, 2011, Promega Corporation, Madison, WI)). Protocols for RNA isolation are disclosed in, e.g., the Maxwell® 16 Total RNA Purification Kit Technical Bulletin (Promega Literature # TB 351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature # TM349, February 2011); the E.Z.N.A. ® FFPE DNA Kit Handbook (OMEGA bio-tek, Norcross, GA, product numbers D 3399-00, D 3399-01, and D 3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D.T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences'HeliScope Gene Sequencing system, or Pacific Biosciences'PacBio® RS platform. In some instances, sequencing may comprise Illumina MiSeq™ sequencing. In some instances, sequencing may comprise Illumina HiSeq® sequencing. In some instances, sequencing may comprise Illumina NovaSeq® sequencing.

Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500 ×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes'rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e−6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 March 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Systems

Also disclosed herein are systems designed to implement any of the methods disclosed herein. In some instances, for example, the system may comprise, e.g., one or more processors, and a memory unit communicatively coupled to the one or more processors and configured to store instructions that, when executed by the one or more processors, cause the system to: receive sequence read data for a plurality of sequence reads obtained from a sample from a subject; identify one or more variant sequences based on the sequence read data; provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and output the pathogenicity prediction score determined for the variant sequence identified in the sample from the subject.

In some instances, the system may comprise, e.g., one or more processors, and a memory unit communicatively coupled to the one or more processors and configured to store instructions that, when executed by the one or more processors, cause the system to: receive sequence read data for a plurality of sequence reads obtained from a sample from a subject diagnosed with a disease; identify one or more variant sequences based on the sequence read data; provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; compare the pathogenicity prediction score determined for the variant sequence identified in the sample from the subject to a predetermined pathogenicity threshold to determine if the variant sequence is pathogenic; and select a treatment for the disease based on a determination that the variant sequence identified in the sample from the subject is pathogenic.

In some instances, the system may comprise, e.g., one or more processors, and a memory unit communicatively coupled to the one or more processors and configured to store instructions that, when executed by the one or more processors, cause the system to: receive sequence read data for a plurality of sequence reads obtained from a sample from a subject; identify at least one variant sequence based on the sequence read data; provide a variant sequence from the one or more identified variant sequences as input to a trained machine learning model configured to determine a cancer type based on the variant sequence and at least one of additional genomic profiling, demographic, or clinical feature data for the sample or subject; and classify the variant sequence as a driver mutation for a gastrointestinal stromal tumor (GIST) based on the variant sequence and determined cancer type.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences'HeliScope Gene Sequencing system, ThermoFisher Scientific's Ion Torrent Genexus system, or Pacific Biosciences'PacBio® RS system.

In some instances, the disclosed systems may be used for predicting the pathogenicity of a variant sequence identified in any of a variety of samples as described herein (e.g., a tissue sample, biopsy sample, hematological sample, or liquid biopsy sample derived from the subject).

In some instances, the plurality of gene loci for which sequencing data is processed to identify variant sequences and/or determine a pathogenicity score for a variant sequence may comprise at least 1, 2, 3, 4, 5, 6, 7, 8, 9, 10, 20, 30, 40, 50, 60, 70, 80, 90, 100, or more than 100 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a pathogenicity score for a variant sequence is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Machine Learning

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, ƒ, where ƒ may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

### Computer Systems and Networks

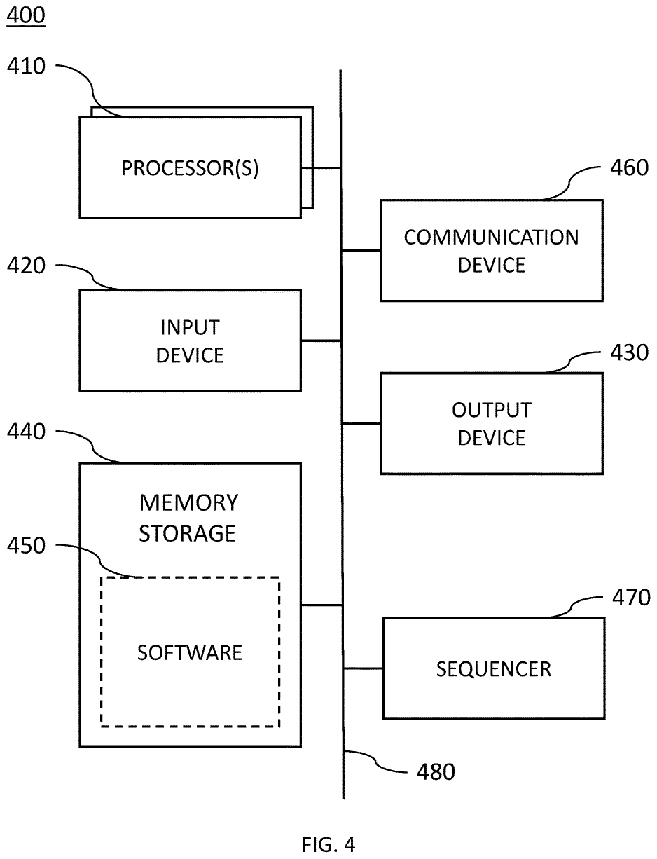

FIG. 4 illustrates an example of a computing device or system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage devices 440, communication devices 460, and nucleic acid sequencers 470. Software 450 residing in memory or storage device 440 may comprise, e.g., an operating system as well as software for executing the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 440 and executed by processor(s) 410, can include, for example, an operating system and/or the processes that embody the functionality of the methods of the present disclosure (e.g., as embodied in the devices as described herein).

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 440, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

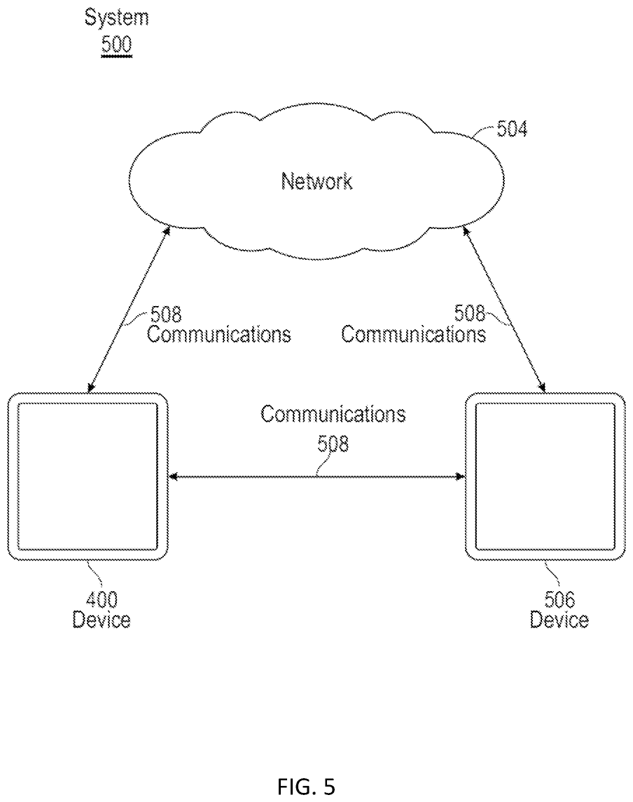

Device 400 may be connected to a network (e.g., network 504, as shown in FIG. 5 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can be implemented using any operating system, e.g., an operating system suitable for operating on the network. Software module 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e. g., processor(s) 410.

Device 400 can further include a sequencer 470, which can be any suitable nucleic acid sequencing instrument.

FIG. 5 illustrates an example of a computing system in accordance with one embodiment. In system 500, device 400 (e.g., as described above and illustrated in FIG. 4) is connected to network 504, which is also connected to device 506. In some embodiments, device 506 is a sequencer. Exemplary sequencers can include, without limitation, Roche/454's Genome Sequencer (GS) FLX System, Illumina/Solexa's Genome Analyzer (GA), Illumina's HiSeq®2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences'HeliScope Gene Sequencing system, or Pacific Biosciences'PacBio® RS system.

Devices 400 and 506 may communicate, e.g., using suitable communication interfaces via network 504, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, Devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 508, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### EXAMPLES

### Example 1—Processes for Assigning Functional Status and Reporting Variant Sequences as Pathogenic

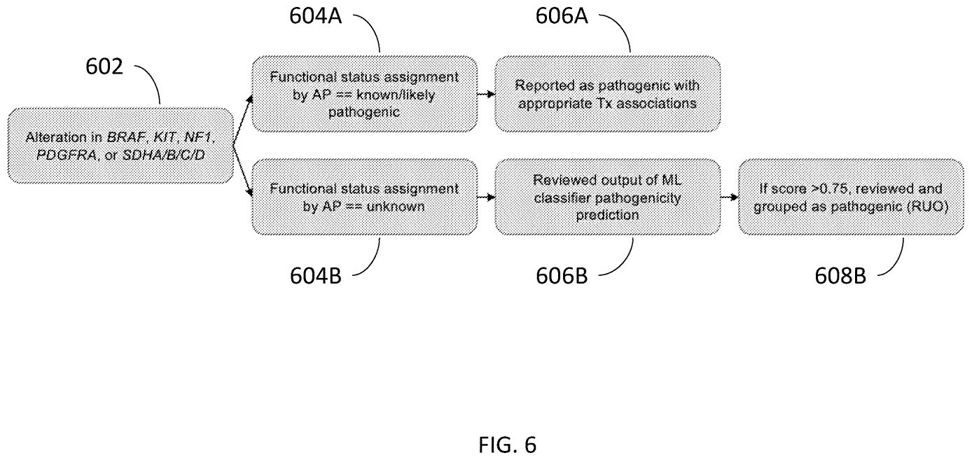

FIG. 6 provides a non-limiting schematic comparison between: (i) a conventional process for assigning functional status and reporting a variant sequence as being pathogenic, and (ii) a novel process for assigning functional status based on the use of a trained machine learning classifier and reporting the variant sequence as being pathogenic.

Steps 602, 604A, and 606A in FIG. 6 illustrate a conventions process for assigning functional status and reporting variant sequences as being pathogenic. At step 602, alterations (also referred to a variants or mutations) in, for example, the BRAF, KIT, NF1, PDGFRA, and/or SDHA/B/C/D genes are identified in sequence read data (e.g., using a variant calling algorithm) derived from a sample from a subject. At step 604A, a functional status assignment is made for each identified variant (e.g., by an annotation pipeline). Variant sequences that are assigned a functional status of “known pathogenic” or “likely pathogenic” are passed to step 606A. At step 606A, “known pathogenic” or “likely pathogenic” variant sequences are reported, along with appropriate therapy associations, in a report provided to a healthcare provider and/or patient.

Steps 602, 604B, 606B, and 608B in FIG. 6 illustrate a novel process for assigning functional status based on the use of a trained machine learning classifier. Again, at step 602, alterations (variants; mutations) in, for example, the BRAF, KIT, NF1, PDGFRA, and/or SDHA/B/C/D genes are identified in sequence read data (e.g., using a variant calling algorithm) derived from a sample from a subject. At step 604B, a functional status assignment is made for each identified variant (e.g., by the annotation pipeline) and variant sequences that are assigned a functional status of “unknown” are passed to a machine learning classifier in step 606B. At step 606B, a variant sequence is processed as input (along with other genomic and/or clinical feature data for the sample) for the machine learning classifier, which then outputs a pathogenicity prediction score. At step 608B, the pathogenicity prediction score is compared to a predetermined cut-off threshold (e.g., 0.75), and variant sequences for which the pathogenicity prediction score are greater than the threshold are assigned a function status of “pathogenic” and reported as such (along with appropriate therapy associations) in a report provided to a healthcare provider and/or patient.

### EXEMPLARY IMPLEMENTATIONS

Exemplary implementations of the methods and systems described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, sequence read data for the plurality of sequence reads obtained from the sample from the subject;
   identifying, using the one or more processors, one or more variant sequences based on the sequence read data;
   providing, using the one or more processors, a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and
   outputting, using the one or more processors, the pathogenicity prediction score determined for the variant sequence identified in the sample from the subject.
2. The method of clause 1, further comprising:
   comparing, using the one or more processors, the pathogenicity prediction score for the variant sequence identified in the sample from the subject to a predetermined pathogenicity threshold, and based on the comparison:
   reporting the variant sequence as being pathogenic if its pathogenicity prediction score is greater than or equal to the predetermined pathogenicity threshold; or
   reporting the variant sequence as being not pathogenic if its pathogenicity prediction score is less than the predetermined pathogenicity threshold.
3. The method of clause 1 or clause 2, wherein the trained machine learning model is further configured to output a prediction of whether the variant sequence is a drug resistance gene.
4. The method of any one of clauses 1 to 3, wherein the subject is suspected of having or is determined to have cancer.
5. The method of clause 4, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms'tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
6. The method of clause 4, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
7. The method of any one of clauses 4 to 6, further comprising treating the subject with an anti-cancer therapy.
8. The method of clause 7, wherein the anti-cancer therapy comprises a targeted anti-cancer therapy.
9. The method of clause 8, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
10. The method of any one of clauses 1 to 9, further comprising obtaining the sample from the subject.
11. The method of any one of clauses 1 to 10, wherein the sample comprises a tissue biopsy sample, a liquid biopsy sample, or a normal control.
12. The method of clause 11, wherein the sample is a liquid biopsy sample and comprises blood, plasma, cerebrospinal fluid, sputum, stool, urine, or saliva.
13. The method of clause 11, wherein the sample is a liquid biopsy sample and comprises circulating tumor cells (CTCs).
14. The method of clause 11, wherein the sample is a liquid biopsy sample and comprises cell-free DNA (cfDNA), circulating tumor DNA (ctDNA), or any combination thereof.
15. The method of any one of clauses 1 to 14, wherein the plurality of nucleic acid molecules comprises a mixture of tumor nucleic acid molecules and non-tumor nucleic acid molecules.
16. The method of clause 15, wherein the tumor nucleic acid molecules are derived from a tumor portion of a heterogeneous tissue biopsy sample, and the non-tumor nucleic acid molecules are derived from a normal portion of the heterogeneous tissue biopsy sample.
17. The method of clause 16, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
18. The method of any one of clauses 1 to 17, wherein the one or more adapters comprise amplification primers, flow cell adaptor sequences, substrate adapter sequences, or sample index sequences.
19. The method of any one of clauses 1 to 18, wherein the captured nucleic acid molecules are captured from the amplified nucleic acid molecules by hybridization to one or more bait molecules.
20. The method of clause 19, wherein the one or more bait molecules comprise one or more nucleic acid molecules, each comprising a region that is complementary to a region of a captured nucleic acid molecule.
21. The method of any one of clauses 1 to 20, wherein amplifying nucleic acid molecules comprises performing a polymerase chain reaction (PCR) amplification technique, a non-PCR amplification technique, or an isothermal amplification technique.
22. The method of any one of clauses 1 to 21, wherein the sequencing comprises use of a massively parallel sequencing (MPS) technique, whole genome sequencing (WGS), whole exome sequencing, targeted sequencing, direct sequencing, or Sanger sequencing technique.
23. The method of clause 22, wherein the sequencing comprises massively parallel sequencing, and the massively parallel sequencing technique comprises next generation sequencing (NGS).
24. The method of any one of clauses 1 to 23, wherein the sequencer comprises a next generation sequencer.
25. The method of any one of clauses 1 to 24, wherein one or more of the plurality of sequencing reads overlap one or more gene loci within one or more subgenomic intervals in the sample.
26. The method of clause 25, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
27. The method of clause 25 or clause 26, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
28. The method of clause 25 or clause 26, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.
29. The method of any one of clauses 1 to 28, further comprising generating, by the one or more processors, a report indicating the pathogenicity prediction score determined for the variant sequence.
30. The method of clause 29, further comprising transmitting the report to a healthcare provider.
31. The method of clause 30, wherein the report is transmitted via a computer network or a peer-to-peer connection.
32. A method for identifying pathogenic variants comprising:
   receiving, at one or more processors, sequence read data for a plurality of sequence reads obtained from a sample from a subject;
   identifying, using the one or more processors, one or more variant sequences based on the sequence read data;
   providing, using the one or more processors, a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and
   outputting, using the one or more processors, the pathogenicity prediction score determined for the variant sequence identified in the sample from the subject.
33. The method of clause 32, further comprising:
   comparing, using the one or more processors, the pathogenicity prediction score for the variant sequence identified in the sample from the subject to a predetermined pathogenicity threshold, and based on the comparison:
   reporting the variant sequence as being pathogenic if its pathogenicity prediction score is greater than or equal to the predetermined pathogenicity threshold; or
   reporting the variant sequence as being not pathogenic if its pathogenicity prediction score is less than the predetermined pathogenicity threshold.
34. The method of clause 32 or clause 33, wherein the trained machine learning model is further configured to output a prediction of whether the variant sequence is a drug resistance gene.
35. The method of any one of clauses 32 to 34, further comprising selecting a treatment for a disease exhibited by the subject based on a pathogenicity prediction score for at least one identified variant sequence that indicates that it is pathogenic.
36. The method of any one of clauses 32 to 35, wherein the one or more identified variant sequences comprise one or more single nucleotide substitutions, one or more short insertions, one or more short deletions, or any combination thereof.
37. The method of any one of clauses 32 to 36, wherein the additional genomic profiling feature data comprises genomic ancestry, microsatellite instability, tumor mutational burden, a determination of somatic versus germline status for the identified variant sequence, or any combination thereof.
38. The method of any one of clauses 32 to 37, wherein the additional demographic feature data comprises the subject's age, sex, race, or any combination thereof.
39. The method of any one of clauses 32 to 38, wherein the additional clinical feature data comprises the subject's sample type, disease diagnosis, family history of disease, or any combination thereof.
40. The method of any one of clauses 32 to 39, wherein the machine learning model comprises a supervised machine learning model.
41. The method of clause 40, wherein the supervised machine learning model comprises a random forest model, a gradient boosted decision tree model, an extreme gradient boosted decision tree model, or a support vector machine.
42. The method of any one of clauses 32 to 41, wherein the trained machine learning model is trained using a training dataset that comprises data for variant sequences identified in samples from a cohort of subjects that includes subjects diagnosed with different diseases.
43. The method of clause 42, wherein the different diseases comprise different cancers.
44. The method of clause 42 or clause 43, wherein the training dataset further comprises additional genomic profiling feature data for the samples from the cohort of subjects.
45. The method of clause 44, wherein the additional genomic profiling feature data comprises genomic ancestry data, microsatellite instability data, tumor mutational burden data, a determination of somatic versus germline status for the identified variant sequence, or any combination thereof.
46. The method of any one of clauses 42 to 45, wherein the training dataset further comprises additional demographic feature data for the cohort of subjects.
47. The method of clause 46, wherein the additional demographic feature data comprises a subject's age, sex, race, or any combination thereof.
48. The method of any one of clauses 42 to 47, wherein the training dataset further comprises additional clinical feature data for the cohort of subjects.
49. The method of clause 48, wherein the additional clinical feature data comprises a subject's sample type, disease diagnosis, family history of disease, or any combination thereof.
50. The method of any one of clauses 32 to 49, wherein the pathogenic prediction score comprises a real number ranging in value from 0.0 to 1.0.
51. The method of any one of clauses 33 to 50, wherein the predetermined pathogenicity threshold has a value ranging from about 0.6 to about 0.9.
52. The method of any one of clauses 33 to 51, wherein the predetermined pathogenicity threshold has a value of about 0.75.
53. The method of any one of clauses 33 to 52, wherein the predetermined pathogenicity threshold is determined on a per-gene basis.
54. The method of any one of clauses 35 to 50, wherein the disease exhibited by the subject is cancer, and the treatment is an anti-cancer therapy.
55. The method of any one of clauses 35 to 54, wherein the disease exhibited by the subject is gastrointestinal stromal tumor (GIST), and the treatment is a tyrosine kinase inhibitor.
56. The method of clause 55, wherein treatment with the tyrosine kinase inhibitor is recommended if the variant sequence is determined to be pathogenic and is not predicted to be a tyrosine kinase inhibitor resistance gene.
57. The method of clause 55, wherein treatment with the tyrosine kinase inhibitor is not recommended if the variant sequence is determined to be not pathogenic or is predicted to be a tyrosine kinase inhibitor resistance gene.
58. The method of any one of clauses 55 to 57, wherein the variant sequence comprises a variant in a BRAF, KIT, NF1, PDGFRA, SDHA, SDHB, SDHC, or SDHD gene.
59. The method of any one of clauses 32 to 58, wherein the sample comprises a tissue biopsy sample or a liquid biopsy sample.
60. The method of clause 59, wherein the sample is a liquid biopsy sample and comprises blood, plasma, cerebrospinal fluid, sputum, stool, urine, or saliva.
61. The method of clause 59, wherein the sample is a liquid biopsy sample and comprises circulating tumor cells (CTCs).
62. The method of clause 59, wherein the sample is a liquid biopsy sample and comprises cell-free DNA (cfDNA), circulating tumor DNA (ctDNA), or any combination thereof.
63. A method for selecting a treatment for a subject in need thereof, the method comprising:
   receiving, at one or more processors, sequence read data for a plurality of sequence reads obtained from a sample from a subject diagnosed with a disease;
   identifying, using the one or more processors, one or more variant sequences based on the sequence read data;
   providing, using the one or more processors, a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject;
   comparing, using the one or more processors, the pathogenicity prediction score determined for the variant sequence identified in the sample from the subject to a predetermined pathogenicity threshold to determine if the variant sequence is pathogenic; and
   selecting a treatment for the disease based on a determination that the variant sequence identified in the sample from the subject is pathogenic.
64. The method of clause 63, wherein the variant sequence is determined to be pathogenic if its pathogenicity prediction score is greater than or equal to the predetermined pathogenicity threshold.
65. The method of clause 63 or clause 64, wherein the variant sequence is determined to be not pathogenic if its pathogenicity prediction score is less than the predetermined pathogenicity threshold.
66. The method of any one of clauses 63 to 65, wherein the trained machine learning model is further configured to output a prediction of whether the variant sequence is a drug-resistance gene.
67. The method of any one of clauses 63 to 66, wherein the additional genomic profiling feature data comprises genomic ancestry, microsatellite instability, tumor mutational burden, a determination of somatic versus germline status for the identified variant sequence, or any combination thereof.
68. The method of any one of clauses 63 to 67, wherein the additional demographic feature data comprises the subject's age, sex, race, or any combination thereof.
69. The method of any one of clauses 63 to 68, wherein the additional clinical feature data comprises the subject's sample type, disease diagnosis, family history of disease, or any combination thereof.
70. The method of any one of clauses 63 to 69, wherein the disease is cancer and the treatment is an anti-cancer therapy.
71. The method of any one of clauses 63 to 70, wherein the disease is gastrointestinal stromal tumor (GIST) and the treatment is a tyrosine kinase inhibitor.
72. The method of clause 71, wherein treatment with the tyrosine kinase inhibitor is recommended if the variant sequence is determined to be pathogenic and is not predicted to be a tyrosine kinase inhibitor resistance gene.
73. The method of clause 71, wherein treatment with the tyrosine kinase inhibitor is not recommended if the variant sequence is determined to be not pathogenic or is predicted to be a tyrosine kinase inhibitor resistance gene.
74. The method of any one of clauses 71 to 73, wherein the variant sequence comprises a variant in a BRAF, KIT, NF1, PDGFRA, SDHA, SDHB, SDHC, or SDHD gene.
75. The method of any one of clauses 63 to 74, wherein the sample comprises a tissue biopsy sample or a liquid biopsy sample.
76. The method of clause 75, wherein the sample is a liquid biopsy sample and comprises blood, plasma, cerebrospinal fluid, sputum, stool, urine, or saliva.
77. The method of clause 75, wherein the sample is a liquid biopsy sample and comprises circulating tumor cells (CTCs).
78. The method of clause 75, wherein the sample is a liquid biopsy sample and comprises cell-free DNA (cfDNA), circulating tumor DNA (ctDNA), or any combination thereof.
79. A method for classifying variant sequences comprising:
   receiving, at one or more processors, sequence read data for a plurality of sequence reads obtained from a sample from a subject;
   identifying, using the one or more processors, at least one variant sequence based on the sequence read data;
   providing, using the one or more processors, a variant sequence from the one or more identified variant sequences as input to a trained machine learning model configured to determine a cancer type based on the variant sequence and at least one of additional genomic profiling, demographic, or clinical feature data for the sample or subject; and classifying, using the one or more processors, the variant sequence as a driver mutation for the determined cancer type based on the variant sequence and the determined cancer type.
80. The method of clause 79, wherein the determined cancer type is a gastrointestinal stromal tumor (GIST).
81. The method of clause 79 or clause 80, wherein the additional genomic profiling feature data comprises genomic ancestry, microsatellite instability, tumor mutational burden, a determination of somatic versus germline status for the identified variant sequence, or any combination thereof.
82. The method of any one of clauses 79 to 81, wherein the additional demographic feature data comprises the subject's age, sex, race, or any combination thereof.
83. The method of any one of clauses 79 to 82, wherein the additional clinical feature data comprises the subject's sample type, disease diagnosis, family history of disease, or any combination thereof.
84. The method of any one of clauses 79 to 83, wherein the one or more identified variant sequences comprise one or more single nucleotide substitutions, one or more short insertions, one or more short deletions, or any combination thereof.
85. The method of any one of clauses 79 to 84, wherein the variant sequence comprises a variant in a BRAF, KIT, NF1, PDGFRA, SDHA, SDHB, SDHC, or SDHD gene.
86. The method of any one of clauses 79 to 85, wherein the classification of the variant sequence as a driver mutation for a gastrointestinal stromal tumor (GIST) is used to diagnose or confirm a diagnosis of GIST in the subject.
87. The method of any one of clauses 79 to 86, further comprising selecting an anti-cancer therapy to administer to the subject based on the classification of the variant sequence as a driver mutation for a gastrointestinal stromal tumor (GIST).
88. The method of any one of clauses 79 to 87, further comprising determining an effective amount of an anti-cancer therapy to administer to the subject based on the classification of the variant sequence as a driver mutation for a gastrointestinal stromal tumor (GIST).
89. The method of any one of clauses 79 to 88, further comprising administering an anti-cancer therapy to the subject based on the classification of the variant sequence as a driver mutation for a gastrointestinal stromal tumor (GIST).
90. The method of any one of clauses 87 to 89, wherein the anti-cancer therapy comprises chemotherapy, radiation therapy, immunotherapy, a targeted therapy, or surgery.
91. The method of any one of clauses 87 to 90, wherein the anti-cancer therapy comprises a tyrosine kinase inhibitor.
92. A method for diagnosing a disease, the method comprising:
   diagnosing that a subject has the disease based on a determination of a pathogenicity prediction score for a variant sequence identified in a sample from the subject, wherein the pathogenicity prediction score is determined according to the method of any one of clauses 32 to 62.
93. A method of selecting an anti-cancer therapy, the method comprising:
   responsive to determining a pathogenicity prediction score for a variant sequence identified in a sample from a subject, selecting an anti-cancer therapy for the subject, wherein the pathogenicity prediction score is determined according to the method of any one of clauses 32 to 62.
94. A method of treating a cancer in a subject, comprising:
   responsive to determining a pathogenicity score for a variant sequence identified in a sample from the subject, administering an effective amount of an anti-cancer therapy to the subject, wherein the pathogenicity prediction score is determined according to the method of any one of clauses 32 to 62.
95. The method of any one of clauses 32 to 62, further comprising determining, identifying, or applying the value of the pathogenicity prediction score for a variant sequence identified in the sample as a diagnostic value associated with the sample.
96. The method of any one of clauses 32 to 62, further comprising generating a genomic profile for the subject based on the determination of the pathogenicity prediction score for a variant sequence identified in the sample.
97. The method of clause 96, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
98. The method of clause 96 or clause 97, wherein the genomic profile for the subject further comprises results from a nucleic acid sequencing-based test.
99. The method of any one of clauses 96 to 98, further comprising selecting an anti-cancer therapy, administering an anti-cancer therapy, or applying an anti-cancer therapy to the subject based on the generated genomic profile.
100. The method of any one of clauses 32 to 62, wherein the determination of the pathogenicity prediction score for a variant sequence identified in the sample is used in making suggested treatment decisions for the subject.
101. The method of any one of clauses 32 to 62, wherein the determination of the pathogenicity prediction score for a variant sequence identified in the sample is used in applying or administering a treatment to the subject.
102. A system comprising:
   one or more processors; and
   a memory communicatively coupled to the one or more processors and configured to store instructions that, when executed by the one or more processors, cause the system to:
      receive sequence read data for a plurality of sequence reads obtained from a sample from a subject;
      identify one or more variant sequences based on the sequence read data;
      provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and
      output the pathogenicity prediction score determined for the variant sequence identified in the sample from the subject.
103. A system comprising:
   one or more processors; and
   a memory communicatively coupled to the one or more processors and configured to store instructions that, when executed by the one or more processors, cause the system to:
      receive sequence read data for a plurality of sequence reads obtained from a sample from a subject diagnosed with a disease;
      identify one or more variant sequences based on the sequence read data;
      provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject;
      compare the pathogenicity prediction score determined for the variant sequence identified in the sample from the subject to a predetermined pathogenicity threshold to determine if the variant sequence is pathogenic; and
      select a treatment for the disease based on a determination that the variant sequence identified in the sample from the subject is pathogenic.
104. A system comprising:
   one or more processors; and
   a memory communicatively coupled to the one or more processors and configured to store instructions that, when executed by the one or more processors, cause the system to:
      receive sequence read data for a plurality of sequence reads obtained from a sample from a subject;
      identify at least one variant sequence based on the sequence read data;
      provide a variant sequence from the one or more identified variant sequences as input to a trained machine learning model configured to determine a cancer type based on the variant sequence and at least one of additional genomic profiling, demographic, or clinical feature data for the sample or subject; and
      classify the variant sequence as a driver mutation for the determined cancer type based on the variant sequence and determined cancer type.
105. The system of clause 104, wherein the determined cancer type is a gastrointestinal stromal tumor (GIST).
106. A non-transitory computer-readable storage medium storing one or more programs, the one or more programs comprising instructions, which when executed by one or more processors of a system, cause the system to: receive sequence read data for a plurality of sequence reads obtained from a sample from a subject; identify one or more variant sequences based on the sequence read data; provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; and output the pathogenicity prediction score determined for the variant sequence identified in the sample from the subject.
107. A non-transitory computer-readable storage medium storing one or more programs, the one or more programs comprising instructions, which when executed by one or more processors of a system, cause the system to: receive sequence read data for a plurality of sequence reads obtained from a sample from a subject diagnosed with a disease; identify one or more variant sequences based on the sequence read data; provide a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject; compare the pathogenicity prediction score determined for the variant sequence identified in the sample from the subject to a predetermined pathogenicity threshold to determine if the variant sequence is pathogenic; and select a treatment for the disease based on a determination that the variant sequence identified in the sample from the subject is pathogenic.
108. A non-transitory computer-readable storage medium storing one or more programs, the one or more programs comprising instructions, which when executed by one or more processors of a system, cause the system to: receive sequence read data for a plurality of sequence reads obtained from a sample from a subject; identify at least one variant sequence based on the sequence read data; provide a variant sequence from the one or more identified variant sequences as input to a trained machine learning model configured to determine a cancer type based on the variant sequence and at least one of additional genomic profiling, demographic, or clinical feature data for the sample or subject; and classify the variant sequence as a driver mutation for a gastrointestinal stromal tumor (GIST) based on the variant sequence and determined cancer type.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for identifying pathogenic variants comprising:
receiving, at one or more processors, sequence read data for a plurality of sequence reads obtained from a sample from a subject;
identifying, using the one or more processors, one or more variant sequences based on the sequence read data;
providing, using the one or more processors, a variant sequence from the one or more identified variant sequences as input to a trained machine learning model configured to determine a pathogenicity prediction score for the identified variant sequence based on the variant sequence and at least one of additional genomic profiling, demographic, or clinical feature data for the sample or subject;
outputting, using the one or more processors, the pathogenicity prediction score determined for the variant sequence identified in the sample from the subject; and
selecting a treatment for a disease exhibited by the subject based on a pathogenicity prediction score for at least one identified variant sequence that indicates that it is pathogenic.

2. The method of claim 1, further comprising:
comparing, using the one or more processors, the pathogenicity prediction score for the variant sequence identified in the sample from the subject to a predetermined pathogenicity threshold, and based on the comparison:
reporting the variant sequence as being pathogenic if its pathogenicity prediction score is greater than or equal to the predetermined pathogenicity threshold; or
reporting the variant sequence as being not pathogenic if its pathogenicity prediction score is less than the predetermined pathogenicity threshold.

3. The method of claim 1, wherein the trained machine learning model is further configured to output a prediction of whether the variant sequence is a drug resistance gene.

4. (canceled)

5. The method of claim 1, wherein the one or more identified variant sequences comprise one or more single nucleotide substitutions, one or more short insertions, one or more short deletions, or any combination thereof.

6. The method of claim 1, wherein the additional genomic profiling feature data comprises genomic ancestry, microsatellite instability, tumor mutational burden, a determination of somatic versus germline status for the identified variant sequence, or any combination thereof.

7. The method of claim 1, wherein the additional demographic feature data comprises the subject's age, sex, race, or any combination thereof.

8. The method of claim 1, wherein the additional clinical feature data comprises the subject's sample type, disease diagnosis, family history of disease, or any combination thereof.

9. The method of claim 1, wherein the machine learning model comprises a supervised machine learning model.

10. (canceled)

11. The method of claim 1, wherein the trained machine learning model is trained using a training dataset that comprises data for variant sequences identified in samples from a cohort of subjects that includes subjects diagnosed with different cancers.

12. The method of claim 11, wherein the training dataset further comprises additional genomic profiling feature data for the samples from the cohort of subjects.

13. The method of claim 12, wherein the additional genomic profiling feature data comprises genomic ancestry data, microsatellite instability data, tumor mutational burden data, a determination of somatic versus germline status for the identified variant sequence, or any combination thereof.

14. The method of claim 11, wherein the training dataset further comprises additional demographic feature data for the cohort of subjects.

15. The method of claim 11, wherein the training dataset further comprises additional clinical feature data for the cohort of subjects.

16. The method of claim 2, wherein the predetermined pathogenicity threshold is determined on a per-gene basis.

17. The method of claim 1, wherein the disease exhibited by the subject is cancer, and the treatment is an anti-cancer therapy.

18. The method of claim 1, wherein the disease exhibited by the subject is gastrointestinal stromal tumor (GIST), and the treatment is a tyrosine kinase inhibitor.

19. The method of claim 18, wherein treatment with the tyrosine kinase inhibitor is recommended if the variant sequence is determined to be pathogenic and is not predicted to be a tyrosine kinase inhibitor resistance gene.

20. The method of claim 18, wherein treatment with the tyrosine kinase inhibitor is not recommended if the variant sequence is determined to be not pathogenic or is predicted to be a tyrosine kinase inhibitor resistance gene.

21. The method of claim 18, wherein the variant sequence comprises a variant in a BRAF, KIT, NF1, PDGFRA, SDHA, SDHB, SDHC, or SDHD gene.

22. The method of claim 1, wherein the sample comprises a tissue biopsy sample or a liquid biopsy sample.

23-34. (canceled)
